(19) United States
(12) Patent Application Publication
(10) Pub. No.: US 2003/0198577 A1
(43) Pub. Date: Oct. 23, 2003

(54) High recovery vial construction

(75) Inventor: Jinghua Schneider, Gurnee, IL (US)

Correspondence Address: BANNER & WITCOFF, LTD., TEN SOUTH WACKER DRIVE, SUITE 3000, CHICAGO, IL, 60606, US

(73) Assignee: Trade Winds Direct, Inc., Gurnee, IL (US)

(21) Appl. No.: 10/126,110

(22) Filed: Apr. 19, 2002

(51) Int. Cl. B01L 3/00; B32B 5/02; B32B 27/04; B32B 27/12; A61B 19/00

(52) U.S. Cl. 422/102

(57) ABSTRACT

A high recovery vial especially useful for collection of powder materials includes a cylindrical passage which terminates with a critically dimensioned frustoconical shaped lower end having arcuate sections connecting the various flat planar walls forming the sides of the cylinder and the frustoconical lower end.

### BACKGROUND OF THE INVENTION

In a principal aspect, the present invention relates to a laboratory vial construction, especially useful for the recovery of powder and/or liquids placed in the vial.

High recovery vials are desired for conduct of laboratory procedures. Such vials are structured with an internal passage that enables maximum recovery of the liquid and/or powder which is placed or stored in the vial.

Heretofore, there were two types of high recovery vials generally known and which were designed primarily to handle liquid materials. The vials generally included a “V” shaped internal bottom surface. A needle could then be inserted into the interior of the vial in the region of the V surface where the liquid would collect resulting in increased amounts of liquid being transferred from the vial into the needle of a pipette, or syringe, for example. Thus, less residue would be left within the vial as contrasted with containers having a flat interior surface. Of course, typically the outside bottom surface of such vials or containers is flat in order that the container or vial might easily sit upon a flat work surface rather than requiring a rack or other support for the vial. Profiles of various alternative vial constructions which comprise prior art are depicted in FIGS. 1, 2 and 3. In FIG. 1, the vial includes a V-shaped lower interior end profile and a flat bottom surface. In FIG. 2 the interior passage has a double planar lower end configuration and a flat bottom surface. FIG. 3 illustrates an alternative V-shaped bottom interior surface. The described vials of FIGS. 1, 2 and 3 are especially useful for recovery of fluid materials. However, they do not work efficiently for both powders and fluids.

FIG. 4 illustrates a prototype vial that includes a high recovery feature, particularly for fluids. In FIG. 4 the internal passage of the vial has reverse incline surfaces so that liquid will collect at the edges or corners of the passage. It further includes a V-shaped center section. Again, such a construction was useful, particularly with respect to liquids, but not highly useful with respect to powders.

Thus, there has remained a need for a vial construction which is useful to provide for high recovery of liquids as well as powders and yet which does not require modification of the external dimensions of typical prior art vials, which will maintain straight sidewalls in the vials and which will also maintain a flat bottom surface for such vials.

### SUMMARY OF THE INVENTION

Briefly, the present invention comprises a high recovery vial construction useful for the recovery of liquids and for the recovery of powders, especially. The vial comprises a tube which has an upper end and a lower end with an internal passage extending from the upper open top end to the lower end. The lower end has a flat outside surface. The inner passage is a straight walled cylindrical passage which has a specially formed lower closure surface that is symmetrical about the center line axis of the passage. The cross sectional profile of the closure surface is comprised of arcuate sections extending from the cylindrical side walls to a flat planar section in the form of a frustoconical surface and which terminates at the center of the passage with an arcuate surface centered on the axis of symmetry of the passage. Choices of the ranges of arcuate surface diameters and incline of the sides of the frustoconical surface are important.

Thus it is an object to provide an improved, high recovery vial especially useful for recovery of powder materials.

Further, it is an object of the invention to provide a vial construction which maintains size characteristics of prior art vials yet which includes the features enabling high recovery of liquids and, in particular, powder materials stored in the vial.

Another object of the invention is to provide a vial construction which can be manufactured using contemporary manufacturing equipment and manufacturing standards inexpensively, yet efficiently.

These and other objects, advantages and features will be set forth in the detailed description which follows.

### BRIEF DESCRIPTION OF THE DRAWING

In the detailed description which follows, reference will be made to the drawing comprised of the following figures:

FIG. 1 is a cross sectional view depicting the profile of a prior art high recovery type vial;

FIG. 2 is a cross sectional profile of a second type of high recovery prior art vial;

FIG. 3 is a cross sectional view depicting a profile of another prior art vial;

FIG. 4 is a further cross sectional view of another vial construction;

FIG. 5 is a cross sectional view of the improved vial construction of the present invention; and

FIG. 6 is an enlarged cross sectional view of the lower end of the vial of FIG. 5 depicting the interior surface of the passage at the lower end of the vial.

### DESCRIPTION OF THE PREFERRED EMBODIMENT

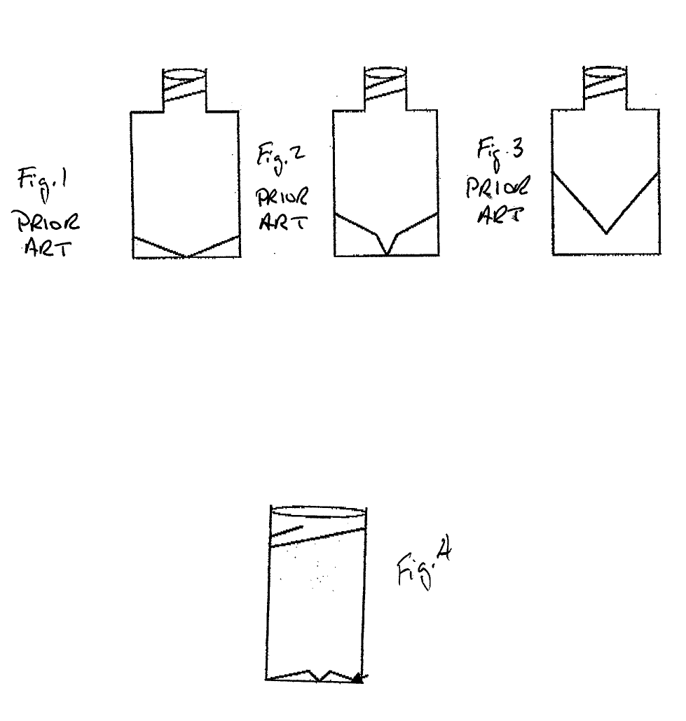

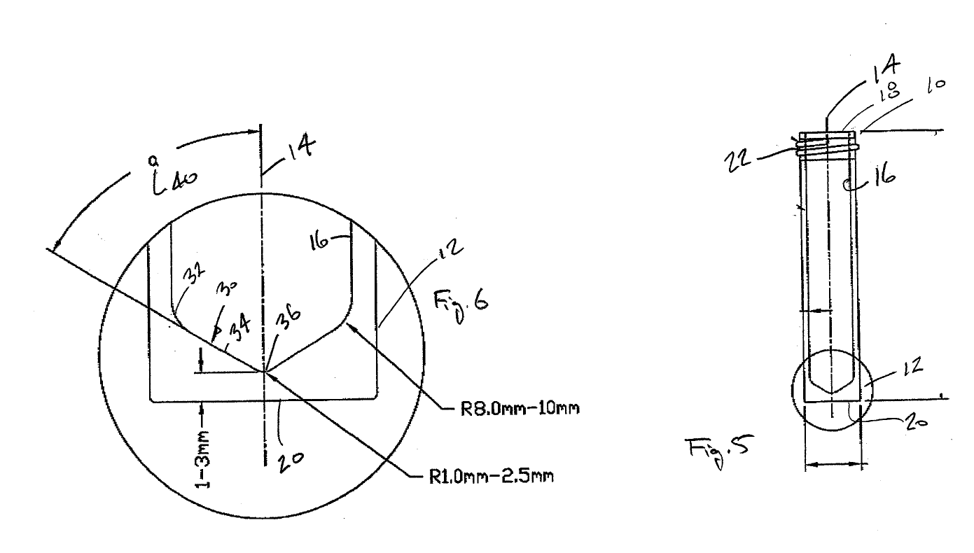

FIGS. 1-4 were discussed in the Background of the Invention. FIGS. 5 and 6 contrast with the construction of FIG. 4, particularly with respect to the construction of the internal surface configuration of the passage within the interior of the vial having a high recovery characteristic.

Referring therefore to FIGS. 5 and 6, the vial depicted includes an upper end 10, a lower end 12, a center line axis of symmetry 14, and an interior straight walled cylindrical passage 16 extending from an open top 18 to the lower end 12. The cylindrical passage 16 thus has an axis of symmetry 14 which extends through the length of the vial. The bottom end 12 of the vial terminates with a flat planar outside support surface 20 that is perpendicular to the axis 14 and enables support of the vial upon a flat surface without placement in a storage rack or the like. The surface 20 is thus a flat planar surface and is generally transverse to the axis 14. The surface 20 may, however, be angled slightly in a manner which continues to enable support of the vial on a flat surface.

The outside of the upper end 10 is optionally threaded, and thus includes threads 22 for receipt of a screw cap. A plug may optionally be placed within the passage 16 at the open top 18 to close the vial.

The vial is typically made from a plastic or glass material depending upon the requirements of the user. The material may be transparent, translucent or opaque. The wall thickness of the vial side wall is typically in the range of 0.8 millimeters to 2.0 millimeters. The typical height of such a vial between the lower end 12 and upper end 10 is in the range of 30 millimeters to 75 millimeters though other heights may be utilized, depending upon the diameter, wall thickness and the like of the vial. Thus, a typical diameter of the vial is in the range of about 10 millimeters to 30 millimeters.

Of significant and critical importance is the construction of the interior of the passage 16 at the lower end. This is illustrated in FIG. 6. As shown in FIG. 6, the cylindrical passage 16 has a uniform diameter along its entire length, including through the open end on top 18, except at the lower end where the passage is formed as a closure surface wall 30 which is symmetrical about the axis 14. The closure wall 30 has a profile which includes a first arcuate section 32 which typically has a radius in the range of about 8 millimeters to 10 millimeters. The first arcuate surface 32 is connected to a straight planar section 34 which defines a frustoconical section of the wall 30. The straight planar section 34 connects to a center arcuate section 36 which acts as a transition between the planar section 34. The center arcuate section 36, which is symmetrical with respect to the axis 14, typically has a radius in the range of about 1 millimeter to 2.5 millimeters and at its apex is in the range of about 1.0 to 3.0 millimeters from surface 20. The planar surface 34 forms an angle 40 with the center line axis 14 in the range of about 30-60° with a preferred angle associated with the material to be stored within the vial. That is, the angle 40 is typically associated with the angle of repose of such materials. A preferred angle of repose is 45°, but again, the angle of repose of the material is important in defining the angle 40.

With the construction as described, recovery of powder materials, especially is enhanced. That is, powder will be guided by the arcuate walls to the center arcuate section 36. Because the angle 40 of the planar wall 34 is associated with the angle of repose of the powder, such powder material will tend to collect at the arcuate center section 36. The smooth interior walls of the cylindrical passage 16 enhance the transport of powder material. Also, because the interior cylindrical passage 16 has a constant diameter, there are no internal shoulders to capture or retain solid powders. Fine powder is otherwise very difficult to remove in the circumstance of a passage having shoulders therein. Of course, the vial may be utilized also with liquid materials with similar advantages. In a most preferred embodiment, the vial is manufactured from a borosilicate glass material. Such a material is typically transparent and is not porous so that it will properly store powder and liquid materials for their maximum recovery.

Certain variations may be employed with respect to the subject matter of the invention. The invention is therefore to be limited only by the following claims and equivalents thereof.

What is claimed is:

1. A high recovery vial comprising, in combination:
a tube having an upper end, a lower end, an internal passage extending from an open top between the upper end to the lower end, said passage having a side wall, said side wall being generally uniformly straight, constant diameter cylindrical wall and extending from the upper end to the lower end and defining a center axis of symmetry, the passage having a closure surface wall at the lower end, said closure surface wall being symmetrical about the center axis and defined by a cross sectional profile through the center axis, said profile consisting essentially of a first arcuate section connected from the cylindrical side wall to a straight planar section at an angle in the range of about 30° to 60° with the axis, to a center arcuate section aligned with the axis, said first arcuate section having a radius in the range of about 8.0 to 10.0 millimeters and said center arcuate section having a radius in the range of about 1.0 to 2.5 millimeters, said lower end having flat planar outside support surface transverse to the axis.

2. The vial of claim 1 wherein the vial has a height in the range of about 30 millimeters to 75 millimeters and a passage diameter in the range of about 10 millimeters to 30 millimeters.

3. The vial of claim 1 wherein the vial is made from a material selected from the group consisting of glass and plastic.

4. The vial of claim 1 wherein the passage is defined by a generally uniform thickness wall.

5. The vial of claim 1 wherein the vial is made from a material selected from the group consisting of transparent materials, translucent materials, and opaque materials.

6. The vial of claim 1 wherein the open top end of the passage is threaded externally.

7. The vial of claim 1 wherein the angle of the planar section is about 45°.
